(19) United States
(12) Patent Application Publication
(10) Pub. No.: US 2003/0211481 A1
(43) Pub. Date: Nov. 13, 2003

(54) Method for identifying cellular targets

(76) Inventors: Albert J. Erives, Berkeley, CA (US); D. Barry Starr, Mountain View, CA (US)

Correspondence Address: ALBERT J. ERIVES, 950 GILMAN DRIVE, SUITE 210, BERKELEY, CA, 94710, US

(21) Appl. No.: 10/142,370

(22) Filed: May 8, 2002

(51) Int. Cl. C12Q 1/68; C12P 19/34; C12N 5/06

(52) U.S. Cl. 435/6; 435/320.1; 435/325; 435/91.2

(57) ABSTRACT

The present invention is directed to nucleic acid constructs and their use in identifying cellular factors that function in various cellular processes involving gene expression. Such factors include those that participate in signaling pathways to regulate cellular gene expression. These factors may be the targets of known therapeutic agents, novel targets for a test compound, or amenable to altered expression to modulate cellular processes.

### CROSS-REFERENCE TO RELATED APPLICATIONS

This application is related to U.S. patent application Ser. No. 09/989,993, filed Nov. 21, 2002, which is hereby incorporated by reference as if fully set forth.

### TECHNICAL FIELD

The present invention is directed to nucleic acid constructs and their use in identifying cellular factors that function in various cellular processes involving gene expression. These factors may be the targets of known therapeutic agents, novel targets for a test compound, or amenable to altered expression to modulate cellular processes.

### BACKGROUND ART

In the pharmaceutical and agricultural industries, high-throughput screening of many chemical compounds is conducted to identify compounds with specific biological effects. A measurable system, known as an assay, is often devised and employed to detect those compounds that might have potential applications. Traditionally, these assays are built around the function of a particular protein target. Therefore, prior to conducting the high-throughput screen, one must first identify a target protein, validate this target protein and then develop a suitable high-throughput assay involving the target protein. This activity is laborious and time-consuming and methods for speeding up this process or bypassing this approach altogether would be advantageous.

One means of identifying targets is to develop cell-based assays. Cell-based assays are said to be open-ended or “black-box” assays. This refers to the fact that a chemical compound applied to the cell-based assay may interact with any number of protein targets, many of which are unknown. Thus, a useful compound identified by such an approach may interact with any number of unknown targets. The positive attribute of such a system is that one can screen against many potential targets without first identifying them. Another positive attribute is that the assay is conducted within the physiologically meaningful context of a cell. However, these types of assays suffer from two negative attributes. The first negative attribute is that it is difficult to optimize the medicinal chemistry properties of a lead compound without first knowing the structure of the target. Also related to not knowing the exact target protein binding the drug, the mechanism of action remains poorly understood. This has consequences not only for optimization, but also for predicting toxic side-effects. The second negative attribute, relates to the non-specificity of the many hits identified by these screens. This latter attribute is partially addressed by building more discrete cell-based reporter assays, such as those described in U.S. patent application Ser. No. 09/989,993, filed Nov. 21, 2001 and entitled, “Regulatory Nucleic Acid Assay for Diagnostic and Library Screens”, which is hereby incorporated by reference as if fully set forth.

Citation of documents herein is not intended as an admission that any is pertinent prior art. All statements as to the date or representation as to the contents of these documents is based on the information available to the applicant and does not constitute any admission as to the correctness of the dates or contents of these documents.

### DISCLOSURE OF THE INVENTION

The present invention provides nucleic acid constructs and methods of using them to identify unknown cellular factors and the molecules with which they interact. The identified cellular factors may be used in assays to screen for compounds that target the factors to produce a biological effect. Such compounds affect the activities of the identified cellular factors to modulate cellular processes in which the factors function. In particular, the invention identifies factors involved in regulating gene expression in various cellular processes.

The invention provides marker/reporter nucleic acid constructs, the expression of which undergoes a detectable change as part of a cellular process. A marker/reporter construct generally comprises a nucleic acid molecule containing a regulatory module operably linked to a basal promoter operably linked to a coding sequence, the expression of which may be readily detected. The regulatory module may be all or part of a naturally occurring regulatory region of a cellular gene. Alternatively, the regulatory module may be a composite of regulatory elements from different regulatory regions. In preferred embodiments of the invention, the regulatory module comprises one or more enhancer or silencer sequences that control activation of the operably linked basal promoter.

The coding sequence preferably encodes a selectable (positive or negative) marker, a detectable reporter, or alternatively, and in a 5′ to 3′ orientation, encodes a reporter followed by a selectable marker. In the last possibility, the sequences encoding both the reporter and the selectable marker are operably linked to the basal promoter. The sequence encoding the selectable marker is also operably linked to an internal ribosome entry site (IRES) situated between the coding sequences for the reporter and the selectable marker. Optionally, the nucleic acid molecule containing an operably linked combination of a regulatory module, basal promoter, and coding sequence is flanked at the 5′, and/or 3′, end by an insulator sequence. In the presence of an insulator sequence, the marker/reporter nucleic acid constructs of the invention may further comprise a second nucleic acid molecule containing a second operably linked combination of a second basal promoter and second coding sequence, optionally with an operably linked second regulatory module. When two regulatory modules are present, the first and second regulatory modules are preferably not identical. The first and second basal promoters may be the same or different but are preferably the same. The first and second coding sequences are preferably different, with the second coding sequence encoding a positive or negative selection marker, a detectable reporter, or a combination of a detectable reporter and a selection marker in an orientation as described above. The insulator sequence(s) prevent effects on the regulatory module of the first nucleic acid molecule from affecting the regulatory module of the second nucleic acid molecule.

The invention also provides a set of expression library constructs capable of expressing nucleic acid molecules of an expression library in combination with the above described marker/reporter constructs. The expression library constructs contain cDNA or genomic sequences such that they may be (individually) expressed in the same cell as that containing a marker/reporter construct as described above. Preferably, the library constructs contain cDNAs prepared from the cell type (cell specific cDNAs) into which the library and marker/reporter constructs are introduced. Alternatively, the library constructs may contain cDNAs prepared from cells that are heterogeneous relative to the cells into which the library and marker/reporter constructs are introduced. In yet another embodiment, the cDNAs may encode a particular type of functionality, such as a receptor, coupling protein, effector, or second messenger. In a further embodiment, the cDNAs may be artificially modified forms of naturally occurring sequences or from cells of a heterologous organism. Use of diverse sources of cDNAs in the present invention permits the identification of a variety of coding sequences able to affect control of the regulatory module of a marker/reporter construct as described herein.

The cDNA of a library construct is operably linked, and thus its expression is controlled by, a constitutive or inducible promoter. The combination of a marker/reporter and one or more library constructs may be referred to as an expression system of the invention.

Cells containing a marker/reporter and an expression library construct of the invention may be assayed for a change in the expression of the marker and/or reporter due to the expression of the library construct. Stated differently, a change in expression of a selectable marker and/or detectable reporter in cells containing an expression system of the invention may be detected and used to identify a library construct as encoding a cellular factor that participates in the control of the regulatory module of the marker/reporter construct. The library construct or the inserted coding sequence may also be isolated from the cell and the sequence of the coding region determined.

The factor may also be identified as participating in the control of the cellular gene(s) from which the regulatory module of the marker/reporter construct was derived. The factor may further be identified as a target for controlling the regulatory module, and thus cellular gene(s). The combination of a marker/reporter construct and an expression library construct in a cell may thus be referred to as an assay system of the invention. The assay system permits the identification of a cellular factor without the need for prior knowledge as to its identity. It is only necessary to have a marker/reporter construct comprising a regulatory module affected by the factor.

Advantageously, the use of the regulatory module to control expression of a selectable marker allows the identification of cellular factors to be based upon a rapid assay based upon cell viability. In an alternative embodiment, use of the regulatory module to control a detectable reporter permits rapid identification of cellular factors to be based upon isolation of cells expressing the reporter by fluorescence activated cell sorting (FACS). Additionally, the optional presence of a second nucleic acid molecule under the control of a different regulatory module allows for the rapid exclusion of cellular factors that non-specifically increase gene expression in a cell.

In one aspect, the assay systems of the invention are used in methods to identify protein factors involved in a signaling pathway that control expression of one or more cellular genes. The methods can be generalized to identify one or more other proteins that interact in a signaling pathway. These protein factors may act directly on a regulatory module, such as by binding directly to it, or indirectly by interacting with one or more other cellular factors that participate in a signaling pathway that controls the regulatory module. Protein factors that activate, or block activation of, a regulatory module such as an enhancer may be identified by detecting expression of a positive or negative selection marker, respectively, or by affecting expression of a detectable reporter. Protein factors that deactivate, or block deactivation of, a regulatory module such as a silencer may be identified by detecting expression of a positive or negative selection marker, respectively, or by affecting expression of a detectable reporter.

The assay systems of the invention may also be used in combination with one or more compounds known to affect, or under consideration as affecting, the control of a regulatory module. In this aspect of the invention, cellular factors are identified by their ability to reverse, or modulate, the action of a compound in an assay system of the invention. Thus a cell containing an expression system of the invention may be contacted with a compound that prevents activation of the regulatory module of the marker/reporter construct such that the cells are not viable (or the reporter is not detectable) unless the expression construct provides a factor that counteracts the inhibitory effect of the compound. This is readily accomplished by use of a coding sequence in the marker/reporter construct that encodes a product that imparts cell viability or is readily detectable upon expression. Conversely, a cell containing an expression system of the invention may be contacted with a compound that activates the regulatory module of the marker/reporter construct such that the cells are not viable (or the reporter is not detectable) unless the expression construct provides a factor that counteracts the activating effect of the compound. This is readily accomplished by use of a coding sequence in the marker/reporter construct that encodes a product that causes cell death (or is readily detectable) upon expression.

Alternatively, a cell containing an expression system of the invention may be contacted with a compound that modulates activation of the regulatory module of the marker/reporter construct such that the cells display a detectably change in viability when the expression construct provides a factor that counteracts the effect of the compound. As a non-limiting example, and with use of a coding sequence in the marker/reporter construct that encodes a product that imparts cell viability in the presence of a cytotoxic agent (such as hygromycin or neomycin), the amount of cytotoxic agent can be titrated such that increases or decreases in cell survival may be detected relative to a change in expression from the marker/reporter construct. Thus with the use of a compound that inhibits activation of the regulatory module of the marker/reporter construct, factors encoded and expressed by an expression construct that tend to decrease (down modulate) the inhibitory effect of the compound may be identified by an increase in cell survival. Conversely, factors that increase (up modulate) the inhibitory effect of the compound may be identified by a decrease in cell survival. All that is necessary is that the amount of cytotoxic agent be titrated so that there is a detectable level of cell survival in the presence of a compound (and absence of expression from an expression construct). Similarly, the present invention may be practiced with use of a coding sequence in the marker/reporter construct that encodes a product that imparts cell lethality in the presence of an agent, such as the combination of a thymidine kinase and gancyclovir. The amount of the agent can be titrated such that increases or decreases in cell survival may be detected relative to a change in expression from the marker/reporter construct. Thus with the use of a compound that inhibits activation of the regulatory module of the marker/reporter construct, factors encoded and expressed by an expression construct that tend to decrease (down modulate) the inhibitory effect of the compound may be identified by an decrease in cell survival. Conversely, factors that increase (up modulate) the inhibitory effect of the compound may be identified by an increase in cell survival. All that is necessary is that the amount of the agent be titrated so that there is a detectable level of cell survival in the presence of the compound and any expression from the marker/reporter construct (and absence of expression from an expression construct).

In yet another embodiment of the invention, a compound known to affect, or under consideration as affecting, the control of a regulatory module may be used in combination with combination with expression library constructs to broadly identify factors that alter the activity of said compound. For example, a compound that causes cell death by inhibiting a signaling pathway that activates expression of an essential cellular gene may be used in combination with the expression library constructs of the invention to identify library constructs encoding factors that reverse the inhibition of the pathway and/or the effects of the compound. The factors encoded by these identified constructs may be the object of further study or compared to the factors identified by use of methods comprising the marker/reporter constructs described above.

### BRIEF DESCRIPTION OF THE DRAWINGS

FIG. 1 is a schematic representation of a transcription unit, such as a gene, comprising various regulatory components.

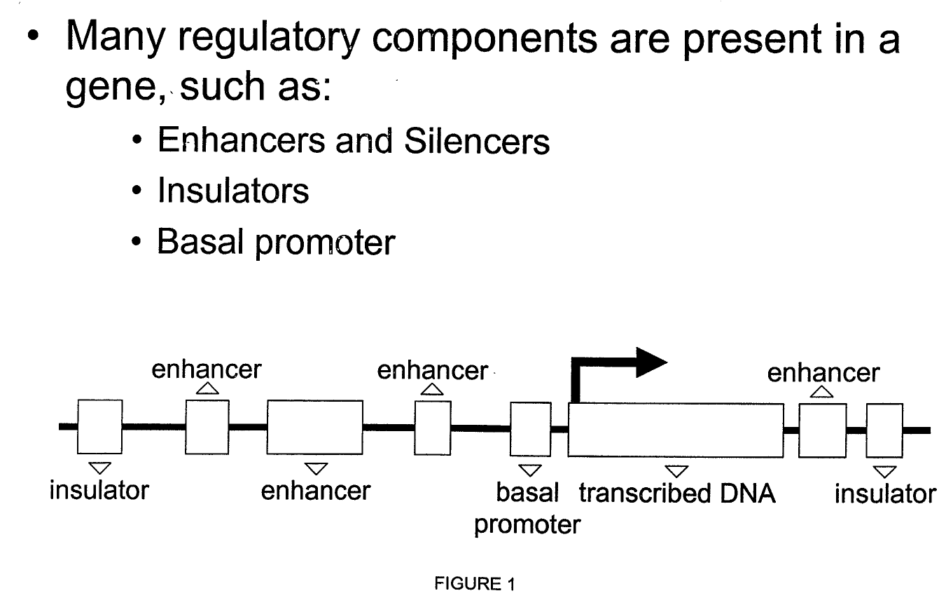

FIG. 2 is a schematic representation of the transcription unit of FIG. 1 with signaling pathways A through D that affect the activity of different enhancers present in the unit.

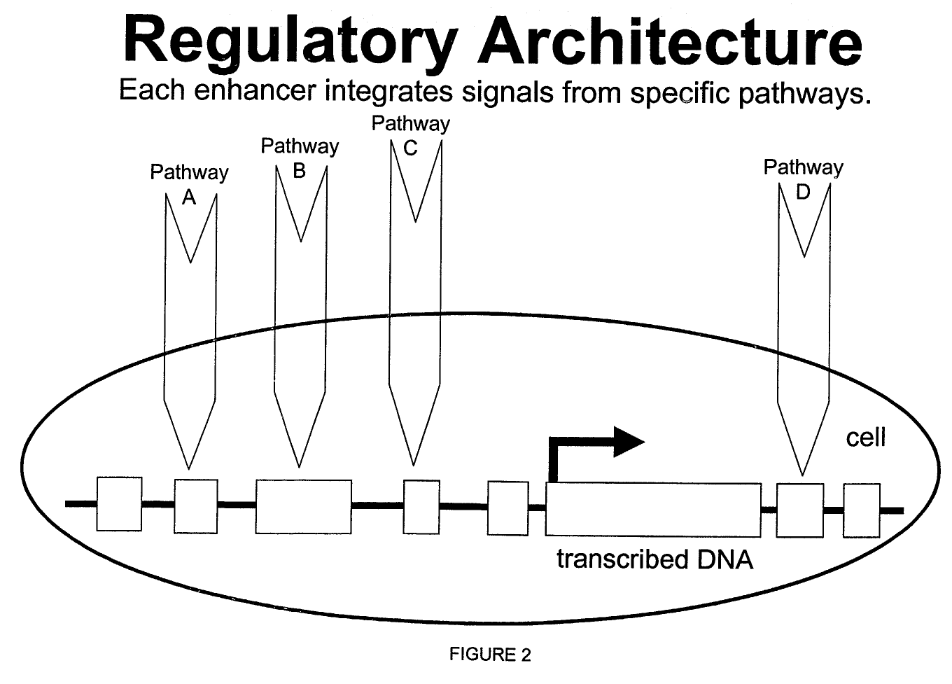

FIG. 3 is a schematic representation of a generalized signal transduction pathway and four variations thereof. Ligands can be extracellular and recognized by a cell surface receptor or membrane permeable such that they interact directly with an intracellular receptor (see pathway 1). Examples of such ligands include steroid hormones which bind to a cytoplasmic receptor. Pathway 2 shows how some receptors, such as certain growth factor receptors, can directly regulate intracellular proteins. Pathway 3 shows how some receptors have intrinsic “effector” capacity to directly produce second messengers. Pathway 4 shows how some second messengers act pleiotropically and interact with a number of target proteins to produce an integrated response. Non-limiting examples of ligands include protease targets, and non-limiting examples of transmembrane receptors include G-protein coupled receptors (GPCRs) and receptor tyrosine kinases (RTKs). Some signaling pathways are also known to involve intracellular signaling proteins, including kinases, and nuclear transcription factors and cofactors, including nuclear hormone receptors.

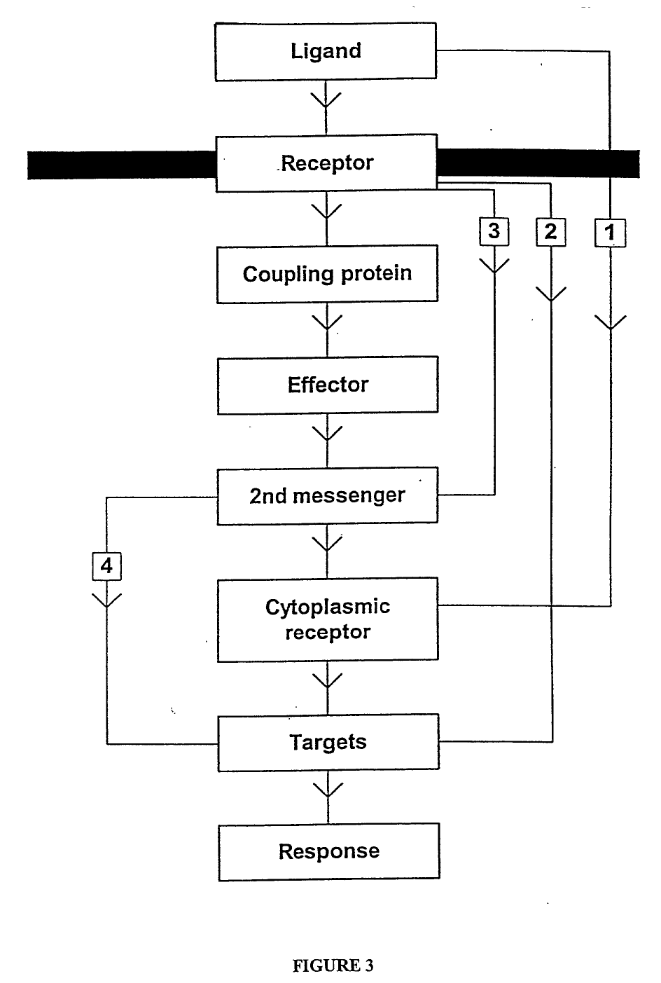

### MODES OF CARRYING OUT THE INVENTION

A marker/reporter construct of the invention comprises a nucleic acid molecule containing a regulatory module operably linked to a basal promoter operably linked to a coding sequence. As used herein, the term “operably linked” refers to a functional linkage between nucleic acid sequences such that a regulatory module functionally regulates the linked basal promoter which functionally controls expression of the coding sequence. A variety of different marker/reporter constructs containing a number of different regulatory modules and different markers and reporters may be prepared and used in the practice of the invention. Marker/reporter constructs containing different regulatory modules and selectable markers can also be used in combination to permit multiplex analysis of the effect of one cellular factor against two different regulatory modules. The markers are preferably heterologous (not normally found in nature) in combination with the operably linked regulatory modules.

Preferably, the construct contains sequences derived from a vector to assist in the propagation and manipulation of the nucleic acid molecule. In some embodiments of the invention the nucleic acid molecule is stably integrated into the genome of a cell in which the invention is to be practiced. The invention includes such cells which may be maintained as a cell line for use in the practice of the invention. Alternatively, the invention may be practiced with use of a marker/reporter construct that is not integrated. Non-limiting examples include stably maintained episomal vectors, such as circular vectors derived from Epstein Barr Virus (EBV), or artificial chromosomes. Vectors that integrate into cellular genomes or are maintained episomally are known in the art and may be used or adapted by the skilled person for use in the practice of the present invention.

Selection of appropriate vectors for propagation or transfer of nucleic acids is well known in the art. The requisite techniques for vector construction, introduction of the vector into the host, and propagation or expression in the host are routine to those skilled in the art. Non-limiting examples of vectors that can be used in the present invention are described below. 100241 The regulatory module generally comprises one or more cis-regulatory sequences that function to regulate the activity of a promoter (or control module). Two general classes of regulatory sequences are enhancers and silencers. An enhancer is an sequence that is present in the genomes of higher eukaryotes and various animal viruses, which can increase the transcription of genes into messenger RNA. Enhancers are often found 5′ to the start site of a gene, and when bound by a specific transcription factor, enhance the levels of expression of the gene, but are not sufficient alone to cause expression. Enhancers can function in either orientation and at various distances from a promoter. Like an enhancer, a silencer can act at a distance from a promoter. But when bound by a trans-acting factor, a silencer suppresses transcription of genes into messenger RNA. Regulatory modules that can be used in the present invention are described below. Additionally, methods for the identification and isolation of additional regulatory sequences and regulatory modules are known in the art. The present invention may be practiced with sequences and modules identified by such methods.

A promoter is a nucleic acid sequence involved in the binding of RNA polymerase to begin transcription. Some promoters contain a consensus region referred to as the TATA box, which is located 5′ from the transcriptional start site of a gene. As used herein, promoter is also referred to as a control module. Examples of control modules for use in the present invention are described below.

The coding sequence preferably encodes a selectable marker, such as, but not limited to, hygromycin resistance, neomycin resistance, Herpes simplex virus thymidine kinase (HSV tk), diptheria toxin A, and aminotriazole (ATZ). Choice of a selectable marker for use in the present invention is readily made by the skilled artisan depending on the embodiment of the invention that is desired. As a non-limiting example, hygromycin and neomycin resistance may be used in embodiments comprising a higher eukaryotic (e.g. mammalian) cell while ATZ can be used in yeast cells where expression of a histidine biosynthesis gene in combination with ATZ results in cell death. Selectable markers for use in the practice of the invention may be generally divided into two groups. The first are positive selection markers that provide resistance (and thus cell viability) against a toxic agent, such as a drug that retards cell growth or is cytotoxic (e.g. hygromycin and neomycin resistance). The second are negative markers that are lethal to a cell upon expression in the presence of an otherwise non-toxic agent (e.g. HSV tk in combination with gancyclovir or a histidine biosynthesis gene in combination with ATZ). Alternatively, the coding sequence encodes a reporter, which is an assayable product upon expression. Examples of reporters for use in the present invention are provided below.

Optionally, the coding sequence encodes both a reporter and a selectable marker, where the reporter and marker are operably linked to an internal ribosome entry site (IRES) situated between the coding sequences for the reporter and the selectable marker such that both may be translated. Stated differently, and in a 5′ to 3′ order, the basal promoter is followed by the coding sequence for a reporter, an IRES, and the coding sequence for a selectable marker. The term “5′” (five prime) generally refers to a region or position in a polynucleotide 5′ (upstream) from another region or position in the same polynucleotide. The term “3′” (three prime) generally refers to a region or position in a polynucleotide 3′ (downstream) from another region or position in the same polynucleotide.

A nucleic acid molecule containing an operably linked combination of a regulatory module, basal promoter, and coding sequence is optionally flanked at the 5′, and/or 3′, end by an insulator sequence. Insulators limit the range of action of regulatory sequences such as enhancers and silencers. Examples of insulators that can be used in the practice of the invention are provided below. Additionally, methods for the identification and isolation of additional insulator sequences are known in the art. The present invention may be practiced with sequences and modules identified by such methods.

In the presence of an insulator sequence, the marker/reporter nucleic acid constructs of the invention may further comprise a second nucleic acid molecule containing a second operably linked combination of a second basal promoter and second coding sequence. The second nucleic acid molecule is also operably linked to the insulator sequence. These second nucleic acid molecules may be used to eliminate changes in transcription of the first nucleic acid molecule that are not specific to the first regulatory module. For example, and without limiting the invention, if a factor expressed by an expression construct of the invention generally increases transcription, such as by being a transcription factor utilized in most or all of a cell's transcription units (e.g. by activation of all basal promoters or all regulatory modules), then it may be identified as activating transcription regulated by the first regulatory module even though it does not act by such a mechanism. Such factors can be eliminated from consideration by use of a second nucleic acid molecule capable of expressing a negative selection marker such that general increases in transcription will result in a lethal phenotype in combination with the nonspecific activation of the first nucleic acid molecule. The ability to utilize such a negative selection marker to eliminate such factors from being identified can be confirmed by using the same basal promoter and/or regulatory module in both the first and second nucleic acid molecules and activating their transcription by use of a factor that activates both.

Expression library constructs of the invention may be prepared by methods known in the art. They are readily prepared by isolation of cDNA or genomic nucleic acid sequences followed by their insertion into an expression vector such that the sequences may be expressed. While most of the discussion herein is with reference to cDNAs, the statements are also readily applicable to genomic sequences unless they would be factually incorrect or identifiable as not pertaining to genomic sequences. Different sets of library constructs may be prepared and used in combination with the marker/reporter constructs of the invention. The library constructs may be prepared with use of a variety of vectors and control modules not limited to those described herein. The control modules may be constitutive or inducible promoters such that the members (or clones) of the library may be introduced into cells transformed (optionally stably transformed) with a marker/reporter construct of the invention. Use of cDNAs prepared from the cells in which the invention will be practiced increases the likelihood of identifying cDNAs encoding cellular factors that normally participate in controlling the activity of a regulatory module. Alternatively, use of cDNAs from heterologous sources or containing modified sequences permits the identification of coding sequences encoding factors that can substitute for naturally occurring cellular factors that participate in controlling the activity of a regulatory module.

Methods for the introduction of nucleic acid constructs into cells are known in the art and include, optionally, the stable integration of the expression construct into a cell. Preferably, the expression constructs are introduced into a population of cells containing a marker/reporter construct of the invention such that on average, each cell contains one expression construct. Stated differently, individual constructs of an expression library are introduced into a population of cells such that each cell, on average, contains one construct of said library. Induction of expression of the coding sequence in the expression construct and contacting the cells with the appropriate selection agent (such as, but not limited to, hygromycin, neomycin, ATZ or gancyclovir) for the marker/reporter construct used permits the identification of coding sequences as participating in control of the regulatory module by simple detection of surviving cells. This may also be viewed as determining whether an individual cell is viable to identify a viable cell as containing an expression construct of interest. Of course the amount of the selection agent must be sufficient to result in cell death, and such amounts are either known or readily determined by the skilled person without undue experimentation.

The coding sequences in the expression constructs in surviving cells may be isolated and identified from the cells by a variety of methods well known in the art. Non-limiting examples include simple PCR mediated by known (expression vector) sequences flanking the coding region in the expression constructs; and isolation of expression library constructs from cells. The coding sequences may also be sequenced by methods known in the art.

The expression constructs of the invention preferably contain cDNAs prepared from. the cells used in the assay system. Alternatively, the constructs may contain cDNAs of known cellular factors having a particular functionality, such as a receptor protein, a coupling protein, a proteinaceous effector or second messenger, or a nucleic acid molecule. These constructs capable of expressing known cellular factors are advantageously used in embodiments of the invention where cells are contacted with compounds known, or thought, to target a particular type of cellular functionality. For example, and without limiting the invention, the use of a compound known, or thought, to inhibit a signaling pathway by inhibiting a kinase molecule may be combined with the use of expression constructs containing coding sequences for various kinases. This provides the ability to identify the actual kinase that is inhibited, if unknown, and/or the ability to identify kinases that functionally substituted for the kinase activity that is inhibited. Alternatively, the invention can be practiced with the use of expression constructs encoding factors, such as second messengers, thought to act downstream of the inhibited kinase activity. This provides the ability to identify factors that act in the same pathway as, or interact with, the inhibited kinase activity.

A cellular factor identified by use of the present invention may be used in assays to screen for compounds that target the factor. The screen may simply utilize a marker/reporter construct of the invention comprising a regulatory module that is responsive to changes in activity of the factor. Such a construct is introduced into a cell and the cell is contacted with test compounds to determine whether they affect the expression of the coding sequence in the construct. Compounds that activate or inactivate the factor may be identified by use of the appropriate marker, and the compounds may thus be used to modulate the cellular pathway(s) in which the factor functions to produce a biological effect in a cell. In particular, the compounds may be used to modulate cellular gene expression controlled by signaling pathways in which the factor functions.

The assay systems of the invention may also be used in combination with one or more compounds known to affect, or under consideration as affecting, the control of a regulatory module. Of course the amount of the compound must be sufficient to affect gene expression controlled by said regulatory module, and such amounts are either known or readily determined by the skilled person without undue experimentation. For example, a compound known to inhibit transcription regulated by a regulatory module may be used in combination with an expression system of the invention to identify members (or clones) of an expression library encoding factors that suppress the inhibition of transcription. This is readily accomplished by use of a positive selection marker the expression of which is needed for cell viability in the presence of a selective agent. Thus only expression constructs encoding a factor that relieves the inhibitory effect of the compound will result in the expression of the positive selection marker and viability of the cells. Expression constructs encoding other factors will not relieve the inhibitory effect and the cells containing them will die. In place of a positive selection marker, a detectable reporter, or a combination of a reporter and a marker, may be used to permit rapid isolation (e.g. by FACS) of cells in which inhibition of transcription regulated by the regulatory module has been suppressed by FACS.

Similarly, the invention may be used in combination with one or more compounds known to activate transcription regulated by a regulatory module. Cells containing a marker/reporter construct comprising a negative selection marker may be contacted with these compounds such that an expression construct expressing a factor which suppresses the activating effect of the compound is needed for cell viability in the presence of an agent that kills the cells when the negative selection marker is expressed. In the absence of such a factor, the compound would activate expression of the negative selection marker and the cells will die. A detectable reporter or a combination of a reporter and a marker, may also be used in this case to permit rapid isolation (e.g. by FACS) of cells in which activation of transcription regulated by the regulatory module has been suppressed by FACS.

Alternatively, the invention may be practiced with a compound known or suspected to have an effect on the control of a regulatory module but without the use of a marker/reporter construct such that expression constructs containing sequences encoding factors that alter the activity of the compound may be identified. This provides a means of broadly identifying factors that affect a compound that affects the control of a regulatory module.

Vectors

As used herein, the term “vector” refers to a nucleic acid molecule capable of transporting another nucleic acid to which it has been linked. One type of vector is an episome, i.e., a nucleic acid capable of extra-chromosomal replication. Other vectors are capable of autonomous replication and/expression of nucleic acids to which they are linked. Vectors may also be used to deliver nucleic acid molecules into a cell for integration into the cellular genome.

Vectors capable of directing the expression of genes to which they are operably linked are referred to herein as “expression vectors.” In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably. In addition, the invention is intended to include other forms of vectors which serve equivalent functions and which become known in the art subsequently hereto.

Vectors can be used for the expression of polynucleotides and polypeptides. Generally, such vectors comprise cis-acting control regions effective for expression in a host operably linked to the polynucleotide to be expressed. Appropriate trans-acting factors either are supplied by the host, supplied by a complementing vector, or supplied by the vector itself upon introduction into the host.

In certain circumstances, the vectors provide for specific expression. Such specific expression may be inducible expression, expression only in certain types of cells, or both inducible and cell-specific. Vectors can be induced for expression by environmental factors that are easy to manipulate, such as temperature and nutrient additives. A variety of vectors such as constitutive and inducible expression vectors for use in prokaryotie and eukaryotic hosts, are well known and employed routinely by those of skill in the art.

A great variety of vectors can be used in the invention. Such vectors include, but are not limited to, chromosomal, episomal, virus-derived (e.g. retroviral, lentiviral, baculoviral, papovaviral, such as SV40, vaccinia virus, adenoviral, fowl pox viruses, and pseudo-rabies virus) vectors, vectors derived from bacterial plasmids, from bacteriophage, from yeast episomes, from yeast chromosomal elements, and vectors derived from combinations thereof, such as those derived from plasmid and bacteriophage genetic elements, such as cosmids and phagemids. Generally, any vector suitable to maintain, propagate or express polynucleotides in a host may be used.

The following vectors, which are commercially available, are provided by way of example. Among vectors for use in bacteria are pQE70, pQE60, and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Eukaryotic vectors available are pWLNEO, pSV2CAT, pOG44, pXT1, and pSG available from Stratagene; and pSVK3, pBPV, pMSG, and pSVL available from Pharmacia. These vectors are listed solely by way of illustration of the many commercially available and well known vectors that are available to those of skill in the art for use in accordance with the present invention. It will be appreciated that any other plasmid or vector suitable for, for example, introduction, maintenance, propagation, and/or expression of a polynucleotide or polypeptide of the invention in a host may be used in this aspect of the invention.

The appropriate DNA sequence may be inserted into the vector by any of a variety of well-known and routine techniques. In general, a DNA sequence for expression is joined to a vector by cleaving the DNA sequence and the vector with one or more restriction endonucleases and then joining the restriction fragments together using T4 DNA ligase. Procedures for restriction and ligation that can be used are well known and routine to those of skill in the art. Suitable procedures in this regard, and for constructing vectors using alternative techniques, which also are well known and routine to those skilled in the art, are set forth in great detail in Sambrook et al. cited elsewhere herein.

The sequence in the vector is operably linked to appropriate expression control sequence(s), including, for instance, a promoter to direct mRNA transcription.

It should be understood that the choice and/or design of the vector may depend on such factors as the choice of the host cell to be transformed and/or the type of protein(s) desired to be expressed. Moreover, the vector's copy number, the ability to control that copy number, and the expression of any other proteins encoded by the vector, such as antibiotic markers, should also be considered. Expression vectors can be used to transfect cells and thereby replicate regulatory sequences and produce proteins or peptides, including those encoded by nucleic acids as described herein.

Regulatory Modules

A regulatory module can comprise an enhancer, silencer, scaffold-attachment region, negative regulatory element, transcriptional initiation site, regulatory protein binding site, any combination or multiplicity of these sequences, and any other regulatory sequence which has a transcription-rate modifying function when placed adjacent to a reporter gene. Such regulatory sequences are described in Goeddel; Gene Expression Technology: Methods in Enzymology 185. Academic Press, San Diego, Calif. (1990).

Examples of enhancer containing sequences are EPO 3′ hypoxia enhancer, cytoplasmic actin promoter, VEGF hypoxia enhancer, LBP-32 enhancer, re1A hypoxia enhancer, PROC hypoxia enhancer, DELTEX hypoxia enhancer, HMOX1 enhancer, GRAP enhancer, BTEγ-4 hypoxia enhancer, CCRdelta5 lymphocyte promoter, and COL4A1. This list is merely an exemplary list of the types of enhancers that can be used in the present invention.

Control Modules

Useful expression control modules can comprise for example, a viral LTR, such as the LTR of the Moloney murine leukemia virus, the early and late promoters of SV40, adenovirus or cytomegalovirus immediate early promoter, the lac system, the trp system, the TAC or TRC system, the T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast alpha-mating factors, the polyhedron promoter of the baculovirus system, and other sequences known to control the expression of genes of prokaryotic or eukaryotic cells or their viruses, and various combinations thereof. Suitable eukaryotic promoters are the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter.

Selection of appropriate promoters for expression in a host cell is a well known procedure. The requisite techniques for introduction of a control module into a nucleic acid construct or vector are routine to those skilled in the art. It will be understood that numerous promoters and other control sequences not mentioned above are suitable for use in this aspect of the invention, are well known, and may be readily employed by those of skill in the art.

In addition, DNA coding a member (or clone) of an expression library can be placed under the control of an inducible promoter, with the result that cells as produced or as introduced into an individual do not express the product but can be induced to do so. Also, a promoter can be a constitutively active promoter.

It should be noted that a control module can be located on the same vector as the regulatory module and/or on a different vector. For example, if needed the control sequence, i.e. promoter, can be “operably linked” to a regulatory module on another vector.

Reporters

A reporter is used to report activated gene expression by providing an easily detectable protein product (e.g., an enzymatic activity such as chloramphenicol acetyl transferase, or CAT). The reporter gene of the present invention can have additional nucleic acids at both ends or at one end of the reporter gene sequence. 100541 Examples of reporters that can be used in the present invention are CAT, lacZ, luciferase (including firefly and Renilla luciferases), Red Fluorescent Protein (RFP) and derivatives thereof, Green Fluorescent Protein (GFP) and derivatives thereof, Blue Fluorescent Protein and derivatives of, Cyan Fluorescent Protein and derivatives thereof, emerald GFP, mGFP5er, Yellow Fluorescent Protein and derivatives thereof, propidium iodide, alkaline phosphatase, or any other detectable enzymatic activity, binding activity, or detectable RNA transcript. Coding sequences for reporters are known in the art or readily obtainable for use in the practice of the invention.

The invention may also be practiced with various forms of GFP that exhibit colors other than green. Additionally, GFP isolated from sources other than the jellyfish Aequorea victoria, such as the sea pansy Renilla reriformis, may be used. As non-limiting examples, the GFPs with GenBank accession numbers U47949 (AGP1); U43284; U36202; U36201; U19282; U19279; U19277; U19276; U19281; U19280; U19278; L29345 (Aequorea victoria); M62654 (Aequorea victoria); and M62653 (Aequorea victoria) may be used. Alternatively, modified GFPs such as AF007834 (GFPuv); U73901 (Aequorea victoria mutant 3); U50963 (Synthetic); U70495 (soluble-modified green fluorescent protein (smGFP)); U57609 (enhanced green fluorescent protein gene); U57608 (enhanced green fluorescent protein gene); U57607 (enhanced green fluorescent protein gene); U57606 (enhanced green fluorescent protein gene); U55763 (enhanced green fluorescent protein (egfp); U55762 (enhanced green fluorescent protein (egfp); and U55761 (enhanced green fluorescent protein (egfp) may be used. GFPs from microorganisms such as U89686 (Saccharomyces cerevisiae synthetic green fluorescent protein (cox3::GFPm-3) gene); and U89685 (Saccharomyces cerevisiae synthetic green fluorescent protein (cox3::GFPm) gene) may also be used in the present invention. Synthetic GFPs such as U87974 (Synthetic construct modified green fluorescent protein GFP5-ER (mgfp5-ER)); U87973 (Synthetic construct modified green fluorescent protein GFP5 (mgfp5)); U87625 (Synthetic construct modified green fluorescent protein GFP-ER (mfgp4-ER)); U87624 (Synthetic construct green fluorescent protein (mgfp4) mRNA)); U54830 (Synthetic E. coli Tn3-derived transposon green fluorescent protein (GF); AAB47853 ((U87625) synthetic construct modified green fluorescent protein (GFP-ER)); and AAB47852 ((U87624) synthetic construct green fluorescent protein) may also be used. Nucleic acids encoding blue fluorescent proteins and identified by the following GenBank accession Nos. may be used: U70497 (soluble-modified blue fluorescent protein (smBFP); 1BFP (blue variant of green fluorescent protein); and AAB16959 (soluble-modified blue fluorescent protein). Similarly, nucleic acids encoding red fluorescent proteins identified by the following GenBank accession Nos. may be used: U70496 (soluble-modified red-shifted green fluorescent protein (smRSGFP); and AAB16958 (U70496) soluble-modified red-shifted green fluorescent protein). Additionally, a fluorophore that changes color with time may be used in the present invention to provide the ability to follow expression over time or determine the approximate time point at which expression occurred. See Teiskikh et al. (Science 290:1585-1588, 2000) for an example of such a fluorophore.

In addition, indirect reporters can be used in the present invention. A secondary protein or compound can be used that interacts with the reporter protein and is labeled with a fluorochrome, radioactivity, or any of the known labeling substances known to one skilled in the art. The secondary protein could be a capture antibody that interacts with the reporter and is coupled to a label.

Excitation and emission maxima for various of the fluorescent proteins and fluorochromes listed above are known in the art.

Insulators

Insulators mark the boundaries of chromatin domains by limiting the range of action of enhancers and silencers. Insulators, which flank many genes, may be responsible for providing a barrier against incursions from surrounding domains. Although the insulator elements vary greatly in their sequences and the specific proteins that bind to them, they have at least one of two properties related to barrier formation. First, insulators have the ability to act as a “positional enhancer blocker.” If the insulator lies between a promoter and an enhancer, then enhancer mediated activation of the promoter is impaired, but if the insulator lies outside the region between enhancer and promoter, little or no effect is observed. Insulators are neutral barriers to enhancer action; they do not inactivate either the enhancer or the promoter.

Second, insulators have the ability to protect against position effects. When genes are removed from their native context, as in transgenic animals, the dominant effect of the new chromosomal environment becomes apparent. Expression levels at the new location often bear no resemblance to that of the gene in its native position. Flanking a transgene with insulators can suppress this variability. Having the ability to protect against position effects and/or to block distal enhancer activity has come to form the operational definition of an insulator. Insulators can act as a modulatable switch, allowing them to function as sophisticated regulatory elements (Bell, A. C., et al., Science, Vol. 29:447-450 (2001).

Examples of insulators that can be used in the present invention are scs, scs', fab7, fab8, the gypsy Su(Hw) array, the cHS4 region from the chick globulin locus, VEGF-A basal promoter region, and the BEAD element. However, other sequences with insulator-like properties may also be used.

If there are multiple transcription units contained in a nucleic acid construct and they are not separated by an insulator, effects on the regulation of one unit can affect regulation of another. An insulator of the present invention can have additional nucleic acids at both ends or at one end of the insulator sequence.

Unless defined otherwise all technical and scientific terms used herein have the same meaning as commonly understood to one of ordinary skill in the art to which this invention belongs.

Having now generally described the invention, the same will be more readily understood through reference to the following examples which are provided by way of illustration, and are not intended to be limiting of the present invention, unless specified.

### EXAMPLE 1

Using Positive Selection and a Compound That Inhibits a Regulatory Module From Activating Expression

The androgen receptor (AR), a nuclear hormone transcription factor that drives expression of the PSA gene, binds androgens as agonists but also binds bicalutamide, an antagonist. It is known that increased levels of AR expression can re-activate AR-dependent gene expression (a model of prostate cancer) in the presence of antagonists such as bicalutamide. A marker/reporter construct containing a first nucleic acid molecule comprising a PSA regulatory module operably linked to a Simian Virus 40 (SV40) basal promoter operably linked to a luciferase coding sequence, an IRES, and a sequence encoding hygromycin resistance is used in combination with a prostate cDNA expression library (in expression constructs of the invention) in an androgen dependent prostate cell line to identify and quantify colonies rescued through AR transgene incorporation (via AR expression from an expression construct of the library). In a first application of the invention, a cell containing the marker/reporter construct and an expression construct containing a member (or clone) of the cDNA library are contacted with both hygromycin and bicalutamide such that expression of hygromycin resistance is necessary for cell viability. Bicalutamide, as an antagonist, acts as the inhibitor of the PSA regulatory module in the marker/reporter construct. Cells able to express hygromycin resistance due to the presence of an expression construct expressing a cDNA that re-activates the PSA regulatory module in the presence of bicalutamide remain viable and selected to identify and/or isolate the cDNA. In addition to AR encoding cDNAs, cDNAs encoding other factors that reverse the inhibitor activity of bicalutamide are identified and/or isolated. In embodiments of the invention using other cDNA expression libraries, such that those of other cell types or from other organisms, factors that are able to substitute for AR are identified via the cDNA encoding them.

This embodiment of the invention may also be practiced in the absence of hygromycin selection but in the presence of bicalutamide, which eventually kills the cells due to their androgen dependency. The cells need not express hygromycin resistance for viability, but only cells containing a cDNA expression construct that restores viability in the presence of bicalutamide will remain viable. These cells may be isolated and the cDNAs they contain isolated and/or identified. In addition to AR encoding cDNAs, cDNAs encoding other factors that reverse the inhibitor activity of bicalutamide, such as, but not limited to, cDNAs that generally increase transcription from the basal SV40 promoter, are identified and/or isolated. A comparison of the results from this and the above use of selection via the marker/reporter construct shows that the fraction of surviving cells that contain an AR transgene is higher when the marker/reporter construct is used.

Of course the above may be practiced with other signaling pathways and compounds that affect them.

### EXAMPLE 2

Using Positive Selection and a Compound That Inhibits a Regulatory Module From Activating Expression: Inclusion of a Second “Control” Construct

The assay system of Example 1 is modified such that the marker/reporter construct also contains a second nucleic acid molecule containing a basal SV40 promoter operably linked to a HSV thymidylate kinase (HSV tk) coding sequence. This second nucleic acid molecule is operably linked to an insulator, which is also operably linked to the first nucleic acid molecule (described in Example 1) such that the PSA regulatory module does not affect expression of the HSV tk coding sequence. Cells containing this construct and expression constructs of an expression library are grown in hygromycin, bicalutamide, and gancyclovir such that cells that specifically express hygromycin resistance without activation of gancyclovir by expression of HSV tk may be selected based upon cell viability. Cells that express HSV tk, such that those containing a cDNA encoding a general activator of transcription from the basal SV40 promoter, will not be selected because they are not viable in the presence of gancyclovir.

This approach will result in a high proportion of the viable cells containing AR encoding cDNAs and is thus able to exclude cDNAs encoding factors that non-specifically act (not through the PSA regulatory module) to increase hygromycin resistance. Advantageously, cDNAs encoding other factors, such as a membrane associated transporter that removes bicalutamide from the cell, will also be identified. This permits identification of factors that associate with bicalutamide. An example of a factor that may be so identified is the mdr-1 gene.

This approach of using a second nucleic acid molecule in the marker/reporter construct may also be applied in the following examples.

### EXAMPLE 3

Using Negative Selection and a Compound That Activates a Regulatory Module Controlling Expression

A marker/reporter construct containing a first nucleic acid molecule comprising a regulatory module operably linked to a Simian Virus 40 (SV40) basal promoter operably linked to a luciferase coding sequence, an IRES, and a sequence encoding HSV tk is used in combination with an expression construct library in cells grown in the presence of gancyclovir. Introduction of a compound that activates the regulatory module to result in expression of HSV tk causes cell death unless expression from an expression construct results in a factor that suppresses the activity of the compound. Use of a second nucleic acid molecule in the marker/reporter construct should include the use of a sequence encoding a positive selection marker to decrease identification of factors that do not act through the regulatory module to affect HSV tk expression.

The surviving cells are isolated, and the sequences responsible for suppressing the activity of the compound, and present in the expression construct, are isolated and/or identified by methods such as PCR or sequencing as described herein.

### EXAMPLE 4

Using Positive Selection and a Compound That Facilitates a Silencer Controlling its Expression

A marker/reporter construct comprising a silencer operably linked to a Simian Virus 40 (SV40) basal promoter operably linked to a luciferase coding sequence, an IRES, and a sequence encoding hygromycin resistance is used in combination with an expression construct library in cells grown in the presence of hygromycin. The cells would not be viable in the presence of a compound that facilitates the silencer's activity unless expression from an expression construct results in a factor that suppresses the activity of the compound such that hygromycin resistance is expressed.

The surviving cells are isolated, and the sequences responsible for suppressing the activity of the compound, and present in the expression construct, are isolated and/or identified by methods such as PCR or sequencing as described herein.

### EXAMPLE 5

Using Negative Selection and a Compound That Deactivates a Silencer Controlling its Expression

A marker/reporter construct containing a first nucleic acid molecule comprising a silencer operably linked to a Simian Virus 40 (SV40) basal promoter operably linked to a luciferase coding sequence, an IRES, and a sequence encoding HSV tk is used in combination with an expression construct library in cells grown in the presence of gancyclovir. Introduction of a compound that deactivates the silencer to result in expression of HSV tk causes cell death unless expression from an expression construct results in a factor that suppresses the ability of the compound to deactivate the silencer. Use of a second nucleic acid molecule in the marker/reporter construct should include the use of a sequence encoding a positive selection marker to decrease identification of factors that do not act through the silencer to affect HSV tk expression.

The surviving cells are isolated, and the sequences responsible for suppressing the activity of the compound, and present in the expression construct, are isolated and/or identified by methods such as PCR or sequencing as described herein.

### EXAMPLE 6

Using Positive Selection With No Compound to Identify Activators of a Pathway

A marker/reporter construct containing a first nucleic acid molecule comprising a regulatory module operably linked to a Simian Virus 40 (SV40) basal promoter operably linked to a luciferase coding sequence, an IRES, and a sequence encoding hygromycin resistance is used in combination with an expression construct library in cells. The regulatory module is such that expression of hygromycin resistance is low or insignificant such that upon addition of hygromycin, the cells would die. Only cells wherein expression from an expression construct results in a factor that activates expression of hygromycin resistance will be viable.

The surviving cells are isolated, and the sequences responsible for activating expression of hygromycin resistance, and present in the expression construct, are isolated and/or identified by methods such as PCR or sequencing as described herein.

### EXAMPLE 7

Using Positive Selection With No Compound to Identify Deactivators of a Silencer

A marker/reporter construct containing a first nucleic acid molecule comprising a silencer operably linked to a Simian Virus 40 (SV40) basal promoter operably linked to a luciferase coding sequence, an IRES, and a sequence encoding hygromycin resistance is used in combination with an expression construct library in cells. The silencer results in no or low expression of hygromycin resistance such that upon addition of hygromycin, the cells would die. Only cells wherein expression from an expression construct results in a factor that deactivates the silencer to result in expression of hygromycin resistance will be viable.

The surviving cells are isolated, and the sequences responsible for deactivating the silencer to permit expression of hygromycin resistance, and present in the expression construct, are isolated and/or identified by methods such as PCR or sequencing as described herein.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. An expression system comprising
a first nucleic acid construct comprising a first nucleic acid molecule comprising a regulatory module operably linked to a basal promoter operably linked to a sequence encoding a selectable marker and
a second nucleic acid construct comprising a sequence encoding a member of an expression library wherein said sequence is operably linked to regulatory elements capable of directing its expression.

2. The system of claim 1 wherein said regulatory module is an enhancer or a silencer.

3. The system of claim 1 wherein said selectable marker is selected from the hygromycin resistance gene, the neomycin resistance gene, and the HSV thymidine kinase gene.

4. The system of claim 1 wherein said first nucleic acid construct further comprises an insulator sequence operably linked 5′ of said first nucleic acid molecule.

5. The system of claim 4 wherein said insulator sequence is selected from Fab-7, Fab-8, scs, scs', and chick HS4.

6. The system of claim 4 wherein said first nucleic acid construct further comprises a second nucleic acid molecule operably linked to said insulator sequence wherein said second nucleic acid molecule comprises a second basal promoter operably linked to a sequence encoding a second selectable marker.

7. The system of claim 6 wherein said second nucleic acid molecule further comprises a regulatory module operably linked to said second basal promoter and said regulatory module is an enhancer or a silencer.

8. The system of claim 6 wherein said second selectable marker is selected from the hygromycin resistance gene, the neomycin resistance gene, and the HSV thymidine kinase gene.

9. The system of claim 1 wherein said member of an expression library is a cDNA clone of a cDNA expression library.

10. A cell or cell line comprising the system of claim 1.

11. A cell or cell line comprising the system of claim 6.

12. A method for identifying a cDNA encoding a product that interacts with components of a signaling pathway that controls the activity of a regulatory module comprising
introducing the system of claim 9 into a cell and allowing the expression of said cDNA;
contacting said cell with an agent that selects for expression of said selectable marker; and
determining the viability of said cell wherein a viable cell is identified as containing a cDNA encoding a product that interacts with components of a signaling pathway controlling the activity of said regulatory module.

13. The method of claim 12 further comprising isolating the sequence of the cDNA from said viable cells.

14. The method of claim 13 wherein said isolating is by PCR amplification of said cDNA from said cells.

15. The method of claim 12 wherein said clones of a cDNA expression library are stably integrated into said cells.

16. The method of claim 12 wherein said clones of a cDNA expression library are expressed upon induction of regulatory sequences operably linked to said cDNA.

17. The method of claim 12 wherein said system of claim 9 is stably introduced into said cell.

18. A method for identifying a cDNA encoding a product that interacts with components of a signaling pathway that is modulated by a chemical compound comprising
introducing the system of claim 9 into a cell and allowing expression of said cDNA;
contacting said cell with said compound to modulate said signaling pathway and an agent that selects for expression of said selectable marker; and
determining the viability of said cell wherein a viable cell is identified as containing a cDNA encoding a product that interacts with components of a signaling pathway modulated by said chemical compound.

19. The method of claim 18 further comprising isolating the sequence of the cDNA from said viable cells.

20. The method of claim 19 wherein said isolating is by PCR amplification of said cDNA from said cells.

21. A method for identifying a cDNA encoding a product that interacts with components of a signaling pathway that controls the activity of a regulatory module comprising
introducing an expression system comprising a first nucleic acid construct into a cell, wherein said construct comprises (i) a first nucleic acid molecule comprising a regulatory module operably linked to a first basal promoter operably linked to a sequence encoding a first selectable marker and (ii) a second nucleic acid molecule comprising a basal promoter operably linked to a sequence encoding a second selectable marker, and wherein said first and second nucleic acid molecules are both operably linked to an insulator sequence such that said regulatory module does not affect expression controlled by said second basal promoter;
introducing a second nucleic acid construct into said cell wherein said construct comprises a cDNA of an expression library wherein said cDNA is operably linked to regulatory elements capable of directing its expression;
allowing expression of said cDNA;
contacting said cell with an agent that selects for expression of said first selectable marker and an agent that selects for expression of said second selectable marker; and
determining the viability of said cell wherein a viable cell is identified as containing a cDNA encoding a product that interacts with components of a signaling pathway controlling the activity of said regulatory module.

22. The method of claim 21 further comprising isolating the sequence of the cDNA from said viable cells.

23. The method of claim 22 wherein said isolating is by PCR amplification of said cDNA from said cells.

24. The method of claim 21 wherein said second basal promoter is the same as said first basal promoter.

25. A method for identifying a cDNA encoding a product that interacts with components of a signaling pathway that is modulated by a chemical compound comprising
introducing an expression system comprising a first nucleic acid construct into a cell, wherein said construct comprises (i) a first nucleic acid molecule comprising a regulatory module operably linked to a first basal promoter operably linked to a sequence encoding a first selectable marker and (ii) a second nucleic acid molecule comprising a basal promoter operably linked to a sequence encoding a second selectable marker, and wherein said first and second nucleic acid molecules are both operably linked to an insulator sequence such that said regulatory module does not affect expression controlled by said second basal promoter;
introducing a second nucleic acid construct into said cell wherein said construct comprises a cDNA of an expression library wherein said cDNA is operably linked to regulatory elements capable of directing its expression;
allowing expression of said cDNA;
contacting said cell with said compound to modulate said signaling pathway, an agent that selects for expression of said first selectable marker, and an agent that selects for expression of said second selectable marker; and
determining the viability of said cell wherein a viable cell is identified as containing a cDNA encoding a product that interacts with components of a signaling pathway modulated by said chemical compound.

26. The method of claim 25 further comprising isolating the sequence of the cDNA from said viable cells.

27. The method of claim 26 wherein said isolating is by PCR amplification of said cDNA from said cells.

28. The method of claim 25 wherein said second basal promoter is the same as said first basal promoter.

29. A method for identifying a cDNA encoding a product that interacts with components of a signaling pathway that controls the activity of a regulatory module comprising
introducing an expression system comprising a first nucleic acid construct into a cell, wherein said construct comprises a nucleic acid molecule comprising a regulatory module operably linked to a basal promoter operably linked to a sequence encoding a fluorescently detectable reporter;
introducing a second nucleic acid construct into said cell wherein said construct comprises a cDNA of an expression library wherein said cDNA is operably linked to regulatory elements capable of directing its expression;
allowing expression of said cDNA;
contacting said cell with an agent that selects for expression of said first detectable reporter; and
identifying a cell as containing a cDNA encoding a product that interacts with components of a signaling pathway controlling the activity of said regulatory module by detecting fluorescence of said reporter in said cells.

30. The method of claim 29 wherein said detection of fluorescence is by fluorescence activated cell sorting (FACS) of said cell.

31. The method of claim 29 further comprising isolating the sequence of the cDNA from said identified cells.

32. The method of claim 31 wherein said isolating is by PCR amplification of said cDNA from said cells.
